(19) United States
(12) Patent Application Publication
(10) Pub. No.: US 2021/0287785 A1
(43) Pub. Date: Sep. 16, 2021

(54) Automatic Sensing for Clinical Decision Support

(71) Applicants: Vanderbilt University, Nashville, TN (US); Oregon State University, Corvallis, OR (US)

(72) Inventors: Daniel Fabbri, Nashville, TN (US); Joseph Coco, Nashville, TN (US); Cheng Ye, Nashville, TN (US); Deirdre Scully, Nashville, TN (US); Candace McNaughton, Nashville, TN (US); Jesse Ehrenfeld, Nashville, TN (US); Christopher Simpson, Nashville, TN (US); Laurie Novak, Nashville, TN (US); Sean Bloos, Nashville, TN (US); Robert Bodenheimer, Nashville, TN (US); Richard Paris, Nashville, TN (US); Julie A. Adams, Corvallis, OR (US); Jamison Heard, Corvallis, OR (US)

(21) Appl. No.: 17/203,204

(22) Filed: Mar. 16, 2021

Related U.S. Application Data
(60) Provisional application No. 62/990,149, filed on Mar. 16, 2020.

(51) Int. Cl. G16H 40/20; G16H 40/67; G16H 15/00; G06K 9/00; G06F 1/16

(52) U.S. Cl. CPC G16H 40/20; G16H 40/67; G16H 15/00; G06F 1/163; G06K 9/00355; G06K 9/00375; G06K 9/00771

(57) ABSTRACT

The present disclosure describes various embodiments of systems, apparatuses, and methods for automated sensing clinical documentation using machine learning. One such method comprises recording a video data feed of a patient being attended to by a medical personnel that is wearing at least one motion sensor; capturing a motion data feed of the medical personnel as the patient is being attended to by the medical personnel; analyzing the collected data feeds to prepare a clinical care record using machine learning algorithms; and transmitting the clinical care record to an upstream healthcare provider. Other methods and systems are also provided.

### CROSS-REFERENCE TO RELATED APPLICATION

This application claims priority to co-pending U.S. provisional application entitled, “Automatic Sensing for Clinical Decision Support,” having Ser. No. 62/990,149, filed Mar. 16, 2020, which is entirely incorporated herein by reference.

### STATEMENT REGARDING FEDERALLY SPONSORED RESEARCH OR DEVELOPMENT

This invention was made with government support under grant number W81XWH-17-C-0252 awarded by the United States Department of Defense. The government has certain rights in the invention.

### BACKGROUND

Healthcare providers face numerous challenges when caring for wounded personnel at a point-of-injury and, appropriately, providing hands-on care is always a higher priority than generating a clinical care record. However, the lack of a usable and portable field clinical care record inhibits communication among medical personnel currently assisting a wounded or injured person, such as a medic team, and upstream healthcare providers, such as a trauma team. These health care providers currently rely on verbal communication and manual recording of information, which are both prone to breakdowns in reliability, usability, and accuracy. Additionally, clinical assessments are influenced by many factors that alter human perceptions of events, which can become even further distorted if events are recorded after initial patient interactions during a patient transport to a healthcare facility (e.g., hospital) and/or handoff to other healthcare personnel. As such, complete situational awareness—the ability to know the full extent of injuries, ongoing and completed interventions, and projected resource needs across multiple simultaneous or staggered injured personnel—is vital in order to maximize survival and minimize long term disability, but nearly impossible with current systems and means of communication.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 provides an overview of an exemplary an automated sensing clinical documentation system (ASCD) system in accordance with various embodiments of the present disclosure.

FIG. 2 is a flowchart of an exemplary automated sensing clinical documentation (ASCD) method in accordance with various embodiments of the present disclosure.

FIGS. 3A-3B shows an application of cardiopulmonary resuscitation on a patient by a medical personnel wearing motion sensors and the resulting sinusoidal acceleration patterns over time with the motion data supplied by the wearable motion sensors, respectively, in accordance with embodiments of the present disclosure.

FIG. 4 illustrates an exemplary video camera layout of an ASCD system in accordance with various embodiments of the present disclosure.

FIG. 5 presents an OpenPose output showing skeletal keypoints including hands, feet, and the head, of a medical personnel performing CPR on a patient, in accordance with various embodiments of the present disclosure

FIG. 6 shows a non-limiting list of classifiable clinical intervention procedures in accordance with various embodiments of the present disclosure.

FIG. 7 shows an injury heatmap that indicates the position of the hands of an attendant medical personnel over a patient's body during a single instance of intubation, insertion of an intravenous catheter, and splinting of a leg (from left to right), in accordance with various embodiments of the present disclosure.

FIG. 8 shows a bar graph representation of the distribution of the hands of a medical personnel over a patient's body during the clinical intervention procedures of a single instance of intubation, insertion of an intravenous catheter, and splinting of a leg (from left to right), in accordance with various embodiments of the present disclosure.

FIG. 9 provides a schematic of a computing device according to various embodiments of the present disclosure.

### DETAILED DESCRIPTION

The present disclosure describes various embodiments of systems, apparatuses, and methods for automated sensing clinical documentation using machine learning by monitoring various video and/or motion data feeds during treatment of a subject requiring or requesting medical assistance. An exemplary method, and/or system consumes video and/or motion data feeds, aggregating contents of the data, such as by synchronizing the feeds and analyzing the feeds to identify patterns or signatures that represent and/or correspond to clinical intervention procedures, and then outputting the detected clinical intervention procedures in a clinical care record (or set of clinical care records) within a storage system that is accessible by other healthcare providers (e.g., via a network dashboard interface) and/or sending the clinical care record(s) to upstream healthcare provider(s) (such that the prepared clinical record can assist in making clinical decisions by the upstream healthcare providers). In various embodiments, motion data feeds may be supplied using wearable technology of an attendant medical personnel or healthcare provider and video data feeds can be provided via cameras that are positioned to capture movements of the medical personnel in relation to the subject/patient, such that an exemplary system/method can automatically sense, document, and transmit clinical events or procedures performed by the attendant medical personnel in clinical care record(s) with little or no manual input (i.e. passive collection of input data (e.g., sensor/video data feeds)) required by the attendant medical personnel. An exemplary an automated sensing clinical documentation system (ASCD) can be used in various operating environments, that include medical transport vehicles, nursing medication facilities, or other type of clinical care facility or space that allows for implementation of system components.

An exemplary clinical care record (or a set of clinical care records) can provide patient clinical status, interventions performed and ongoing, and anticipated needed resources upon arrival and/or handoff at an upstream healthcare provider (e.g., hospital), without requiring active input from personnel in the field, which can overcome human misperceptions and error, provide high fidelity and reliable data, and/or allow communication across multiple patients and providers at the same time.

Given the challenges of using traditional technologies to document clinical care at the point-of-injury, such systems/methods can ensure better, more consistent, and clear communication among care teams. In various embodiments, an exemplary system can passively collect data from a combination of wearable motion sensors (e.g., inertial measurement units (IMUs) (such as accelerometers) and/or electromyography (EMG) sensors) and video cameras, such that a computing device (employing machine learning, object detection, activity recognition, and summarization algorithms) can be configured to analyze the collected data to construct clinical care record(s) for the patient or subject. In various embodiments, an exemplary system can be configured to transmit clinical care record(s) to a trauma team prior to patient arrival or handoff.

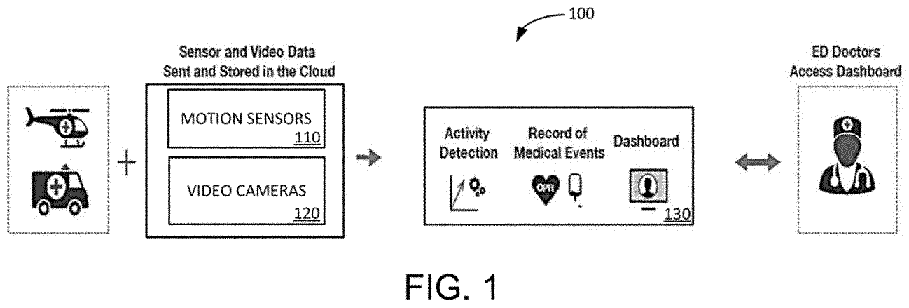

FIG. 1 provides an overview of an exemplary an automated sensing clinical documentation system (ASCD) system 100 in accordance with various embodiments of the present disclosure. The exemplary system includes one or more wearable motion sensors 110 and one or more video cameras 120 that are positioned to capture motion and video data streams during treatment of a patient or subject by an attendant medical personnel. Certain equipment for the automated sensing clinical documentation system can be transportable within the field and housed in a carrying case. For example, an exemplary carrying case may be capable of storing EGM and/or IMU armbands, smart watches having EGM and/or IMU units (e.g., accelerometers); smart phones having IMU units and configured to communicate with the smart watches and/or other sensors (e.g., via Bluetooth communications); WiFi hotspot device(s) that are configured to communicate with a computing device 130, peripheral cameras 120, sensors 110, and/or other communication devices; respective charging devices, and a laptop device to allow for manual reporting or logging of data by the attendant medical personnel or technician (“medic”) in the field, if desired, and may also be coupled to one or more sensors and/or WiFi hot spot. In various embodiments, connections with the computing device 130 may be provided via a virtual private network (VPN) connection. In various embodiments, the computing device 130 is located remotely from the wearable sensors 110 and video cameras 120, such as a cloud server.

In various embodiments, the wearable motion sensor(s) 110 are to be worn by one or more attendant healthcare provider(s) that are attending to the patient or subject and the video camera(s) 120 are positioned to capture a video data stream of an upper torso and/or hands of the attendant(s) while attending the patient. The respective data streams are provided as input to one or more computing device(s) 130 that are configured to perform activity recognition methods to identify the clinical events or procedures being performed by the attendant(s) and the sequence and/or timing at which they are performed, record the clinical events in a clinical care record (or a set of clinical care records) that is accessible via a network interface (e.g., web dashboard) to upstream healthcare providers for review. In various embodiments, an exemplary ASCD system 100 can be deployed in transport vehicles to collect sensor and video data simultaneously during patient transport and supplied to an upstream healthcare provider facility (e.g., hospital) before the transport vehicle arrives at the facility, upon arriving at the facility, or soon thereafter (depending on network availability for sending the clinical care record(s) during transport).

In various embodiments, the computing device(s) 130 (e.g., a cloud server) can utilize various object detection and activity recognition algorithms to analyze collected sensor data and video feeds to determine the type of clinical intervention procedures are performed by the attendant and the associated timing at which they are performed. Corresponding, in various embodiments, an exemplary network interface, such as a web-based dashboard, is presented by the computing device(s) 130 to display the prepared clinical care record(s) so that receiving physicians can analyze the collected data and prepare for patient arrival. In an exemplary embodiment, the network interface can display clinical care records that include an injury heat map, a triage score, and a list of performed interventions (with associated confidence levels). Accordingly, upstream healthcare personnel, such as emergency department physicians, can evaluate the automatically generated clinical documentation in the form of the clinical care records and the information presented in the network interface for a quality assessment of the status of the patient.

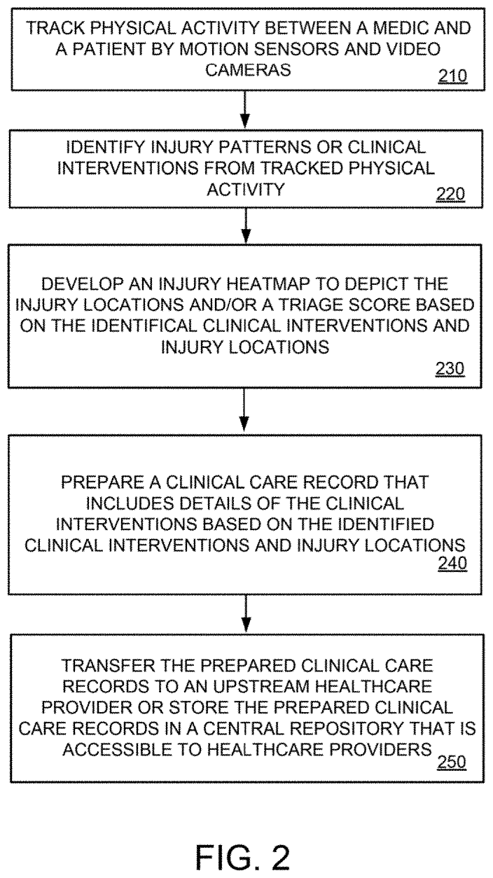

Referring now to FIG. 2, a flowchart of an exemplary automated sensing clinical documentation (ASCD) method is presented. First, in block 210 of the flowchart, physical activities between a medical personnel and a patient are tracked by motion sensors and/or video cameras that are positioned to capture motion and video data streams of clinical procedural interventions as performed by the medical personnel to a patient/subject. In various embodiments, motion sensors in the form of IMU units and/or EMG sensors are, but not limited to only being, worn by the attendant and cameras are located in a transfer vehicle to capture the motion and/or video data streams. Next, in block 220, the tracked physical activity (via capture data streams) are used to identify injury patterns and/or clinical interventions (e.g. performing a patient assessment, applying pressure to a wound, hanging an intravenous (IV) bag or blood bag, application of hemostatic dressings, use of a limb tourniquet, insertion of an airway, placement of a needle thoracostomy, performing CPR (Cardiopulmonary Resuscitation), intravenous line starts, etc.) by one or more computing device(s) 130 using machine learning. For example, hand and body positions of the attendant with respect to the patient can be used to identify the injury locations and clinical interventions.

For example, in various embodiments, video data can be used to track the position of the medical personnel's hands over the patient's body using a computer vision system software, such as OpenPose. In such systems/methods, the medical personnel's hand position can act as a prior function to determine the possible procedures that can be performed at a given point in time. Additionally, wearable sensor data (IMU and EMG data) can be measured and subsequently summarized with various metrics (e.g., entropy, power, etc.). These data feeds may then be fed into classifiers to predict what intervention procedure is being performed, if any, by the medical personnel. In certain embodiments, over twenty different clinical intervention procedures can be recognized using wearable sensors and video data. As such, wearable sensors can obtain measured sensor data (e.g., a medical personnel's arm movements and muscle contractions), and the video data can be used to localize the medical personnel's hand positions, relative to a patient, in order to determine an active body region or on which body part the medical personnel is performing a procedure. Determining the active body region of the patient culls the number of potential procedures to recognize, as certain procedures are only performed in specific body regions (i.e., placing an oral airway only occurs near the patient's head). This class set reduction can improve clinical procedure recognition accuracy.

Additionally, in block 230, an injury heatmap diagram is developed to depict the injury locations and/or a triage score is assigned to the patient, and in block 240, a clinical care record is prepared that includes details of the clinical interventions, the triage score that is computed based on the identified clinical interventions and injury locations, and/or the injury heatmap diagram. The prepared clinical care records may then be transferred to an upstream healthcare provider or stored in a central repository that is accessible to healthcare providers, as shown in block 250. In various embodiments, the prepared clinical care records can be merged with other generated care records and can be confirmed, modified, or deleted by the medical personnel attendant (before/after being transferred) or the upstream healthcare provider (after being transferred).

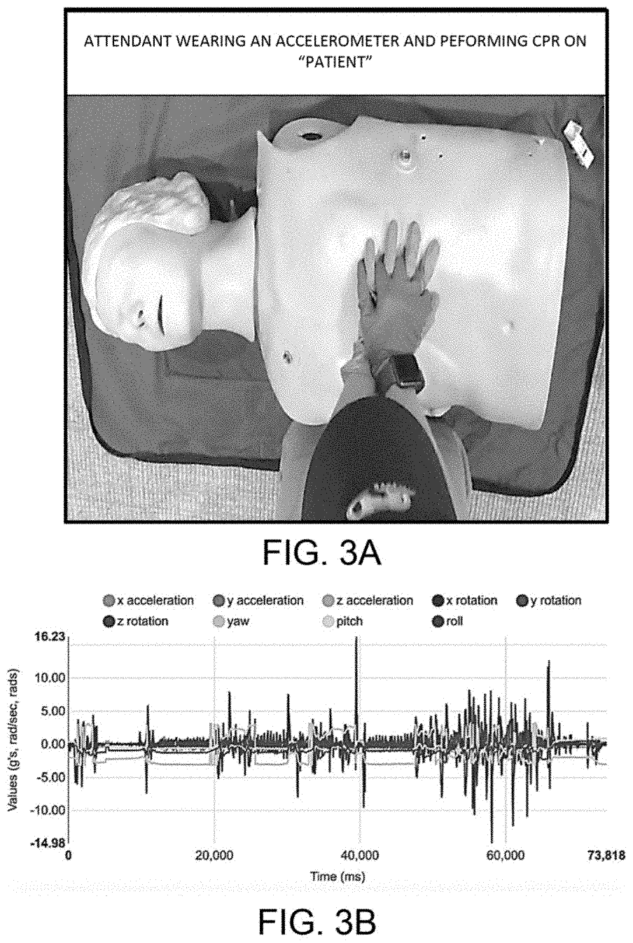

In various embodiments, various wearable motion sensing devices can be deployed, such as a smart watch having an accelerometer that is configured to collect wrist acceleration, rotations and yaw, pitch and roll and/or EMG sensors that is configured to measure muscle contractions. Accordingly, IMU units and/or EMG sensors, such as those contained within arm wearable device (e.g., smart watches or fitness trackers that can be worn on one or both arms of a medical personnel attendant), can track motion, determine elevation changes, and durations of activities of the medical personnel attendant. Thus, in various embodiments, smart watches can be configured to send sensor measurements to the computing device(s) 130 for plotting and analysis of the sensor data to correlate with recorded video data and detect task activity of the attendant using machine-learning methods. For example, a CPR event can be detected based on particular sinusoidal acceleration patterns over time of the motion data supplied by the arm wearable device, as demonstrated in FIGS. 3A-3B. Accordingly, many key clinical events, which are important to both document and communicate upstream, can be detected passively using data from these sensors, without active user input by the medical personnel attendant.

As a medical personnel attendant moves their hands in specific sequences or patterns when performing clinical interventions, wearable accelerometers attached to the wrists and/or the hip or upper arm of attendants can collect sufficient signal to automatically detect the type of procedure being performed based on the signal pattern or signature over time of the motion sensing data. Dependent on the type of activity, a single wrist sensor when combined with either a hip based sensor or a second upper arm sensor has been shown to provide reliable activity detection. In various embodiments, the use of three or more wearable accelerometers is deployed at various locations of the body of the medical personnel attendant to detect and recognize the intervention activities of the attendant. For example, certain actions can be reliably detected using an accelerometer sensor placed on a hip of the medical personnel attendant, while different actions cannot be reliably detected without an accelerometer sensor placed near the attendant's hands. For example, when a hip-positioned sensor is used (e.g., a smartphone placed in a pocket of the medical personnel attendant), activities, such as changing a bed, putting on gloves, measuring a pulse) can be identified, while activities that predominately use the hands requires an arm- or wrist-positioned sensor to be used. Since many smart watches are paired with a smartphone, using the smartphone in the pants pocket may be a viable option for improving detection accuracy. In certain embodiments, it is possible that the hip based sensor may not provide sufficient information to differentiate the desired activities, thus, a sensor placed on the upper arm is deployed instead.

In various embodiments, additional sensor(s) besides motion-based and video-based sensors can also be integrated, such as sound-based environment sensors that capture audio signals made during the monitored intervention activities of the medical personnel. Further complicating the sensor measurements are external accelerations associated with implementations involving a patient transport vehicle. For example, helicopters and ambulances experience external environmental forces that will induce acceleration on the medical personnel attendant and patient. To mitigate the external acceleration noise during data collection, in various embodiments, a motion sensor can be mounted on the transport vehicle so that vehicle acceleration measurements can be subtracted from the medical personnel's sensor data for analysis by the computing device(s) 130. Correspondingly, various sensors can provide raw data that will be preprocessed and filtered to remove noise and artifacts (such as the gravity vector from IMU data) by the computing device 130. In various embodiments, the results are decomposed into time windows that can range from 2 to 30 seconds or longer. The update rate of the chosen sensors can directly impact the required time window. Further, short time windows have proven appropriate for simple repetitive behaviors, but longer windows are required for more complex, less repetitive behaviors.

The placement of sensors on subjects and/or vehicles can be limited by real-word constraints. For example, a medical personnel must be able to perform their clinical responsibilities and cannot be distracted by a device or have their range of motion limited, and mounting positions of video cameras may be limited by the size of the transport vehicle and position of people and equipment in it. Body cameras are an alternative to vehicle-mounted cameras but must also not limit functionality of the medical attendant. In general, exemplary systems/methods of the present disclosure utilize a multi-modal approach of sensing data including: (x, y, z)-accelerations, (x, y, z)-rotations, yaw, pitch, and roll of the attendant's hands over time, and (x, y, z)-positions of the attendant's hands over time in addition to video data.

As discussed, video cameras can provide valuable information about personnel activity and hand position over the patient's body without active user input. While accelerometers are able to track the intensity and direction of motion, they do not provide exact location data. Given that a specific injury location (e.g., torso vs. extremity) is likely to be an important care element under certain conditions, more complex information can be gathered from monitoring an attendant's hand position over the patient's body (such as if they are holding a bandage on a wound or performing a jaw thrust). Therefore, the combination of video data with accelerometer data can provide for more reliable intervention detection than accelerometers alone in many scenarios (e.g., if the hands are not positioned over the patient's head, it is unlikely the attendant is intubating the patient). Thus, hand tracking may be used in correlation with accelerometer data to detect interventions.

In certain embodiments, video sensors or camera are positioned within an operational environment, such as an interior of a transport vehicle, (e.g., statically mounted video and/or depth cameras on a ceiling or perimeter of a transport vehicle). Video or RGB (red, green, blue) cameras capture video images in normal visual visible spectrum, whereas depth cameras usually operate in the infrared spectrum and broadcast a pattern, which a camera sensor then resolves into a depth image sequence that is read like a video sequence. Overhead or side view statically mounted depth and video cameras may provide larger scale contextual information, which may be useful for certain recognition algorithms. For example, recognition algorithms using static cameras often rely heavily on human pose estimation from the video data, and sometimes this can be computationally demanding. Data from the various video sensors can be combined into a data vector to be processed by activity recognition algorithms of the computing device(s) 130.

Note that certain operational environments (e.g., ambulance) may be well-lighted with a standard layout, which provides a good environment for vision algorithms to work in using a single modal video approach (e.g., video cameras), while certain operational environment (e.g., a MEDEVAC helicopter) may be likely to be more challenging due to potentially poorly lit or pitch-black working environments, necessitating a multi-modal video approach involving video and depth cameras.

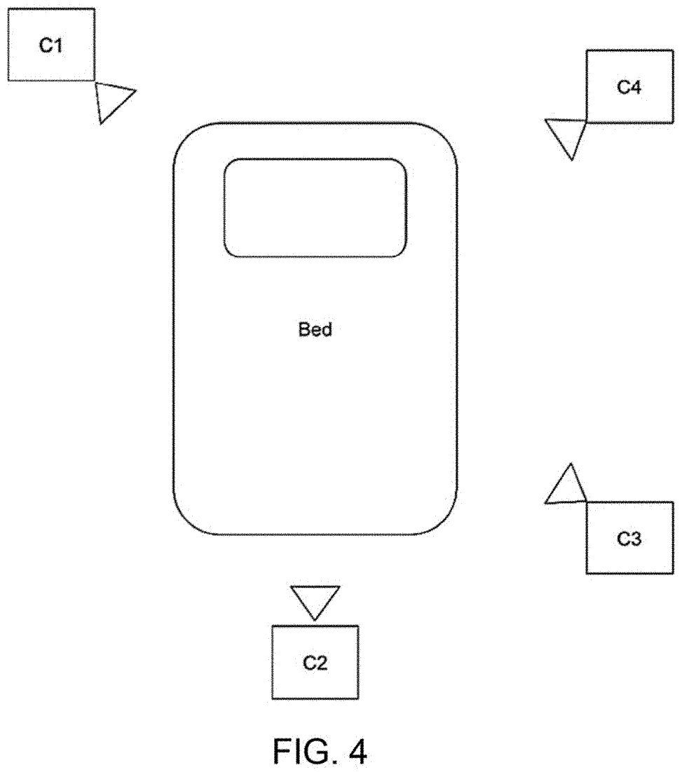

FIG. 4 illustrates an exemplary video camera layout for various embodiments. The positioning of the cameras relative to a bed or gurney/stretcher (upon which a patient is placed) is shown in the figure, in which cameras (e.g., four cameras in one non-limiting embodiment) are arranged at varying angles with respect to the patient. Accordingly, video data can be collected from four angles for 3D reconstruction. In one embodiment, each of these cameras are at a height of 2 meters to ensure that the patient and subject are visible in each camera. An individual camera (e.g., Camera 2 (C2)) can be selected that is able to capture the patient's body centered in the frame so that screen space may roughly correspond to a 2D plane directly over the body. In one embodiment, the cameras are equipped to record at a 3840 by 2160 pixel resolution at 24 frames per second. In various embodiments, the respective cameras generate a series of 181 second videos with one second of overlapping frames between clips in the series. The final one second can be removed (24 frames) of overlapping video so that no duplicate processing is completed by a computer vision system deployed by the computing device(s) 130 (e.g., OpenPose software). Accordingly, in certain embodiments, each 3 minute video is analyzed with OpenPose, which executes on a computing device implementing an NVidia Docker virtual machine using two GeForce GTX Titan X GPUs.

Although OpenPose uses only a single camera angle to generate its 2D representations, an exemplary video camera layout has viewpoints from multiple cameras. While additional perspectives do not provide additional information for the purpose of training using the OpenPose position tracking, multiple cameras do provide the ability to secure information in the event of occlusion, as when the medical personnel attendant blocks the view of the patient from one camera. Even more so, active body region detection method can be improved by incorporating multiple camera angles, as a 3D representation of the attendant's hands is feasible. Multiple camera angles may be less sensitive to object occlusion (i.e., the attendant is blocking a camera view) or confusion. For example, a body region detection method is sensitive to limitations of the OpenPose skeleton keypoints, as the keypoints are a sparse representation of a human body. In various embodiments, an exemplary machine learning algorithm may be trained using the two closest body parts for each hand in order to better estimate the active body region.

In addition to assist in identifying the clinical intervention procedures performed by a medical personnel attendant, an exemplary visual processing system can be used to identify the area(s) of the patient's body that is (are) injured. Such information can be used to communicate an injury location to upstream healthcare providers. In various embodiments, information describing the attendant's hand position over the patient's body can be automatically included in the care record as an injury heat map to indicate areas of injury. For example, in certain embodiments, the injury heat map can be constructed by measuring the time the attendant's hands are above specific quadrants of the human body over transport time and supplied as part of clinical care records to the upstream provider.

After the initial raw data collection from the various motion sensors and video sensors/camera, the data is analyzed by the computing device(s) 130 to automatically detect clinical interventions and produce clinical care record(s). Image and signal processing methods are performed to identify commonly occurring patterns that correlate with interventions and injuries, which can be documented electronically in a clinical care record and transmitted to upstream healthcare providers.

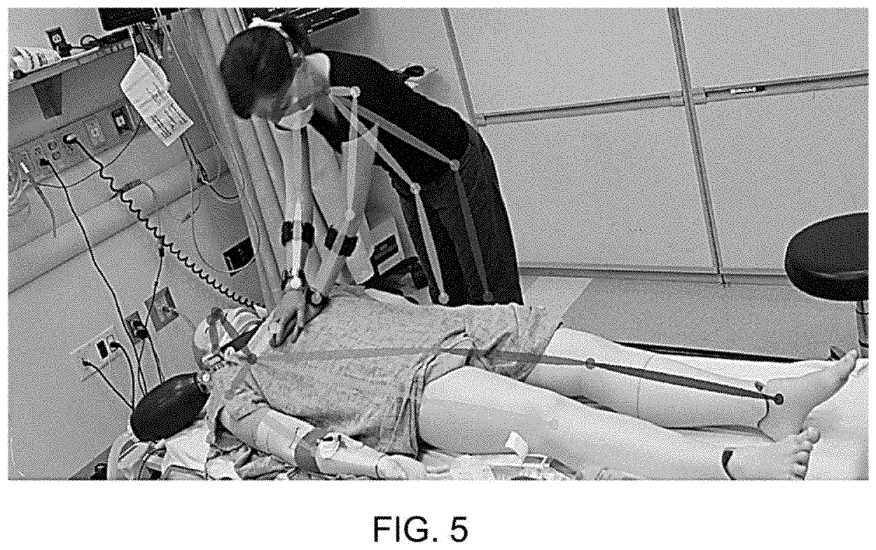

In various embodiments, an exemplary computing device 130 uses sensor data feeds to identify clinical intervention procedures. Additionally, video feeds can be processed with a computer vision system (e.g., OpenPose) that analyzes video frames to identify people in the respective frame and calculate their skeletons. In an exemplary embodiment, the skeletons include 18 different key point positions including hands, feet, and the head, which designate where in each frame the people (e.g., attendant(s) and patient) and their extremities are, as illustrated in FIG. 5. Given these key points, an exemplary computing device 130 can identify the patient using simple heuristics such as being centered in the frame and having minimal movement. Next, the exemplary computing device 130 can identify the attendant as the person closest to the patient. Once the patient and the attendant are identified, the exemplary computing device 130 can construct a ‘patient space,’ which is a geometric space relative to the patient's body and track the attendant's hands in the patient space (i.e., hands over the head or over the leg).

Next, in various embodiments, a specific activity or feature extraction routine of the computing device(s) 130 identifies activity characteristic feature vectors that are used to identify intervention activities. For example, in various embodiments, a sensor-equipped arm band (e.g., a Myo device) is worn on each of a medical personnel attendant's forearms and captures arm movements and muscle contractions via an inertial measurement unit (IMU) and an 8-channel electromyography (EMG) sensor, respectively. Acceleration and orientation data can be captured at 50 Hz, while the EMG data is captured at 200 Hz. In various embodiments, the sensor-equipped arm band automatically calculates the IMU's roll, pitch, and yaw, and a 5 second window, with a 1 second stride, is applied to each sensor signal. Alternative window sizes may be utilized in various embodiments.

In certain embodiments, each sensor signal's mean, standard deviation, and max value are calculated for each window and are typical features extracted for activity recognition, whereby each sensor signal is transformed into the frequency domain using the fast Fourier transform in order to calculate the signal's spectral entropy. Thus, four features can be extracted from each sensor signal resulting in fifty-six features per medic hand, in various embodiments.

An orthogonal approach to classification using wearable sensor data is to use image processing to track the medical personnel attendant's hands during the clinical intervention procedures. Since many procedures are localized to certain areas on a patient's body, making relative hand location an enticing factor, the image-based hand localization system determines the patient's closest limb to the attendant's hands for a particular procedure and uses that information for classifier refinement. To do so, computer vision system software, such as, but not limited to, OpenPose, can be utilized. For example, OpenPose is an image-based human body pose detection framework that generates skeletal keypoints using a COCO (Common Objects in Context) system in screen space pixel coordinates for both the medical personnel and the patient (in which OpenPose parameters can be tuned to accommodate a prone individual).

During an exemplary detection procedure, assuming the medical personnel's hands are proximal to the patient eliminates the need for 2D to 3D image conversion. Thus, the calculated distance between the medical personnel's hand keypoints and each skeleton keypoint on the patient is in pixel space. This measurement's variability and noise is reduced by averaging the limb position over 1 second 24 frames) in order to determine the patient's closest limb to the medical personnel's hands per second. The closest limb is mapped to one of four body regions: head, chest, arm, or leg.

For clinical intervention procedure classification, extracted features from the IMU and EMG sensors are fed into a random forest classifier, which is a supervisory-based machine-learning algorithm that is an ensemble of individually trained decision tree classifiers. The random forest classifies a signal by taking the class mode of the decision tree ensemble. In one embodiment, 100 decision trees with a max-depth of 500 are used, where the parameters are chosen based on classifier performance. The targeted domain requires knowing if a procedure was performed, not that every single window is correctly classified. Assuming a clinical intervention procedure's start and stop time is known, the procedure can be classified as the majority vote of each classified window within the procedure time frame. For example, if CPR (Compressions) consists of fifteen windows where ten windows are classified correctly and the other five windows are not, then the procedure can be correctly classified as CPR. Algorithm 1 (below) provides exemplary pseudo code for this classification. In particular, the algorithm cycles through each window between the procedure start and stop time, extracting features from the wearable sensor data for each window. DetermineBodyRegion( ) runs OpenPose on the window's image data and determines the window's active body region, which is used to determine which trained random forest classifier to apply. The extracted features are fed into the classifier to predict a clinical procedure for the window. After each window is processed, the algorithm returns the Majority Vote of the predicted procedures using Max(ProcedureCount( )). Accordingly, in general scenarios, the majority vote method may perform better in real-world scenarios over the random forest method.

Algorithm 1 Clinical Procedure Classification Algorithm

Input: Procedure Start/Stop Time, Wearable Sensor Data,

Video Data

Output: ProcedureClassification

PredictedProcedureList = [ ]
for each window between Procedure Start and End time
do

Features = ExtractFeatures(window,

WearableSensorData)

ActiveBodyRegion = DetermineBodyRegion(window,

Video Data)

Classifier = DetermineClassifier(ActiveBodyRegion)
Procedure = Classifier.Predict(Features)
PredictedProcedureList.append(Procedure)

end for
return Max(ProcedureCount(PredictedProcedureList))

Data collection involving a larger training set can allow for a more sophisticated approach to clinical intervention procedure detection. In such embodiments, a larger training set can allow for deep learning algorithms to be applied, where features can be learned from the wearable sensor data using convolutional neural networks, rather than being selected a priori. Additionally, in certain embodiments, a long short-term memory recurrent architecture can be applied to the convolutional neural network to better capture the time-dependencies that occur within a procedure. Combining deep-learning techniques with the active body detection and majority vote methods can improve the performance of an exemplary automated sensing clinical documentation system substantially, in certain scenarios.

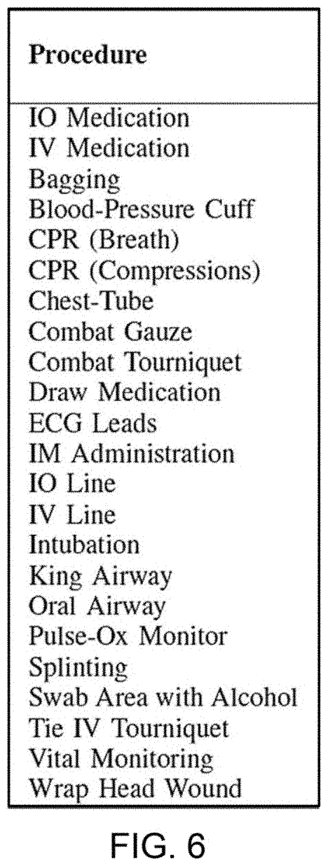

Typically, in various embodiments, the feature vectors and ground truth data are used to train a machine learning or neural network of the computing device(s) 130 in order to develop scores representative of specific activities. The resulting scores may then be used to recognize the activity classes using new data. Given the domain complexity and the actions to be recognized, which have no clearly defined start and stop times, task models can be developed and used to train the computing device(s) to improve activity recognition rates when processing, filtering, and analyzing the input data. For example, a task model can involve breaking down a certain tasks performed by a medical personnel attendant into fine-grained descriptions of the performance of the tasks and associated subtasks that can include detailed descriptions of actions performed by the attendant's hands, the location of these actions in relation to the patient's body and in relation to the artifacts in an operating environment space, (e.g. an IV rack mounted on the wall or ceiling), variations in the actions that are related to individual practitioners, spatial constraints, or environmental conditions (e.g., lighting, noise), and relationships among the tasks. Accordingly, such task models can be leveraged by the activity recognition routines of an exemplary computing device 130. Exemplary task models can cover various intervention procedures, such as, but not limited to, IV start, chest compressions, intubation, insertion of chest tube, taking vital signs, administration of blood, preparing and administering tranexamic acid, performing a neurologic exam, observation of hypotension, etc. A non-limiting list of such classifiable intervention procedures are shown in FIG. 6.

In addition to identification of clinical intervention procedures and related details, the computing device(s) 130 can determine injury location(s) based on the video and/or motion data streams. As such, an injury heat map, a triage score, and a list of performed interventions (with associated confidence levels) can be generated as clinical care records. In general, the injury heatmap may indicate the portions of the patient's body that the medical personnel attendant is focused on, and the triage score may provide a categorization of a status of the patient based on attendant activity levels.

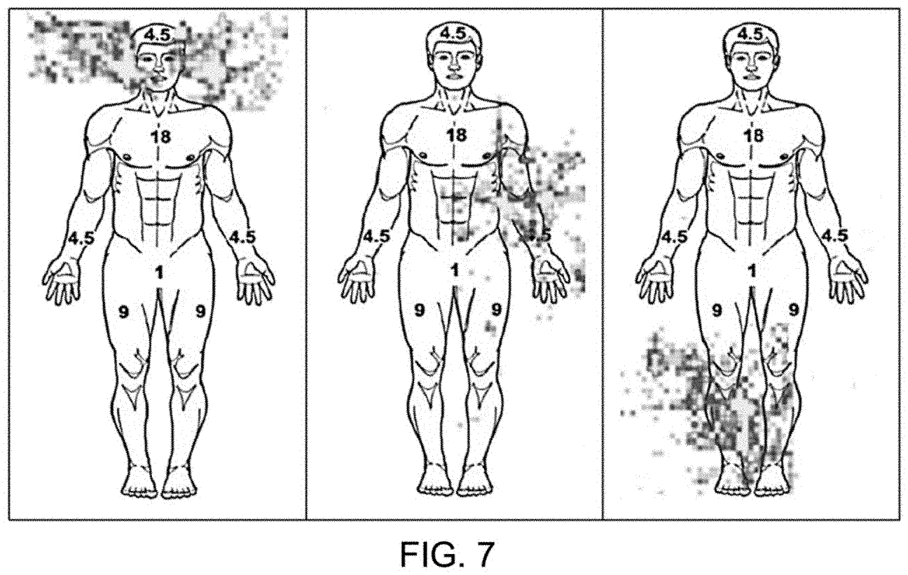

For example, given intervention procedure-specific video windows or segments, hand key points of the attendant can be extracted and used to generate a Gaussian field around them. This extraction process is done for every frame and summed over all frames in the window. By summing intensities of the fields over all windows and frames, an injury heatmap can be generated over the body showing the most frequently occurring positions of the hands for the window, as represented by FIG. 7. In particular, the injury heatmap illustrates the position of an attendant's hands over the patient's body during a single instance of intubation, insertion of an IV, and splinting of a leg (from left to right). Such injury heatmaps can be used as training data for a neural net (convolutional neural network (CNN) or deep neural network (DNN)) classifier, which is configured to classify intervention procedures. In general, different colors can be used to represent a positioning and duration of an attendant's hands over the patient's body. For example, a yellow color may represent the areas above and around the patient where the attendant's hands are located most often and a different color may represent the areas where the medical personnel attendant's hands are located less often.

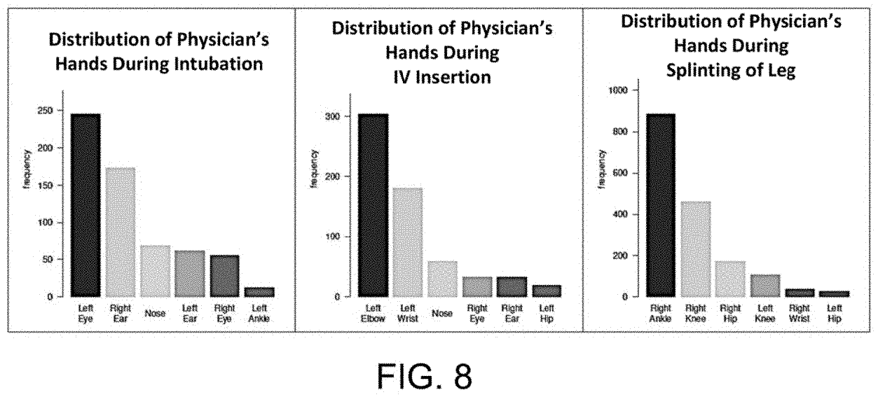

Exemplary injury heatmaps represent one way to visualize the location of injuries of a patient's body as reflected by the position of a medical personnel's hands relative to the body during an intervention procedure. An additional visualization method that can be provided with the clinical care record(s) are bar charts that show how much time a medical personnel's hands spend in close proximity to each of the skeletal points of the patient (as constructed by computer vision system software, such as OpenPose, implemented by an exemplary computing device(s) 130). To create the bar charts, the closest skeletal point of the patient to each hand position of the metric is calculated in addition to the distance using pixels as reported by OpenPose. Accordingly, this data can be used separately or in conjunction with the heatmap data as training data for a CNN classifier. Correspondingly, FIG. 8 shows bar chart distributions of the hands of a medical personnel over a patient's body during the clinical intervention procedures of a single instance of intubation, insertion of an IV, and splinting of a leg (from left to right).

As previously stated, a triage score (in addition to an injury heat map and/or a list of performed interventions (with associated confidence levels)) can be generated as part of the clinical care records by embodiments of the present disclosure. It is noted that existing triage scoring systems provide broad categorization of patient status, which make it difficult to differentiate between patients with the same score but different injury sets. A finer-grained triage scoring would allow for more efficient patient care, especially in cases of mass traumas. Thus, in accordance with various embodiments, triage scores of the present disclosure can narrowly categorize specific activity levels and duration of attendant activity and be supplemented with clinical procedural details which can inform receiving providers of patient injury severity. With these summarizations, trauma teams can better gauge the severity of a patient's injuries and prepare for multiple patient arrivals. Correspondingly, an exemplary computing device 130 can predict a triage scored based on input sensor data. One exemplary approach, among others, to calculate the triage score from sensor data can be based on average accelerations per second during transport, the max acceleration during transport, and the minimum time between ‘high’ levels of accelerations.

After generation of the various clinical care records by an exemplary computing device 130, the clinical care records can be sent to upstream healthcare providers for analysis and/or stored in a central repository that is available to the upstream healthcare providers. In various embodiments, a data transmission system, such as 4G wireless connections or Wi-Fi network connections, can be used to transfer the prepared clinical care records. However, it is noted that real-world settings may limit transmission system availability (e.g., FAA regulations, network interruptions, network unavailability, etc.). In the case when no transmission system is available, clinical care data may be transmitted on arrival or handoff. In general, electronic clinical care records, in accordance with the present disclosure, are designed to be generated in real time, or near real time, and transmitted upstream to healthcare providers as a supplement or in place of a verbal handoff in order to increase the accuracy and detail of clinical information transmitted to upstream clinical providers and teams, particularly in high-acuity and trauma settings.

In general, previous attempts to develop portable documentation systems for use in the field have largely failed for a variety of reasons including poor usability and the requirement to pause ongoing care activities. Regarding conventional combat military practices, the lack of a usable and portable field health care record inhibits communication among combat military medical personnel working on the battlefield and upstream healthcare providers. These teams currently rely on verbal communication and manual recording of information, which are both prone to breakdowns in reliability, usability, and accuracy. Providers face numerous challenges when caring for wounded personnel at the point-of-injury and, appropriately, providing hands-on care is always a higher priority than generating a clinical care record. Additionally, clinical assessments are influenced by many factors that alter human perceptions of events, which can become even further distorted if events are recorded after patient transport. Additionally, because information is rarely provided in advance of patient arrival, receiving teams are typically more focused on time-sensitive tasks (e.g. patient movement, exposure, stabilization, primary trauma survey) rather than receiving a report from the field personnel. These challenges hinder medical teams in all settings from optimizing care—especially in combat settings where communications channels, time and resources are limited.

In comparison, benefits of embodiments of the present disclosure include automatic generation of a clinical care record and streamlined communication of patient information from the field that reduces miscommunication, increase the readiness of receiving teams, and decreases overall patient complications. Development of a system to automatically generate and transmit a clinical care record will bridge the gap between current communication and documentation practices so that information can flow seamlessly and in real-time across settings of care. In accordance with various embodiments of the present disclosure, supplementing existing communication methods with an automatically produced list of clinical intervention procedures with time stamps has the potential to more adequately prepare for the triage and downstream management of trauma cases.

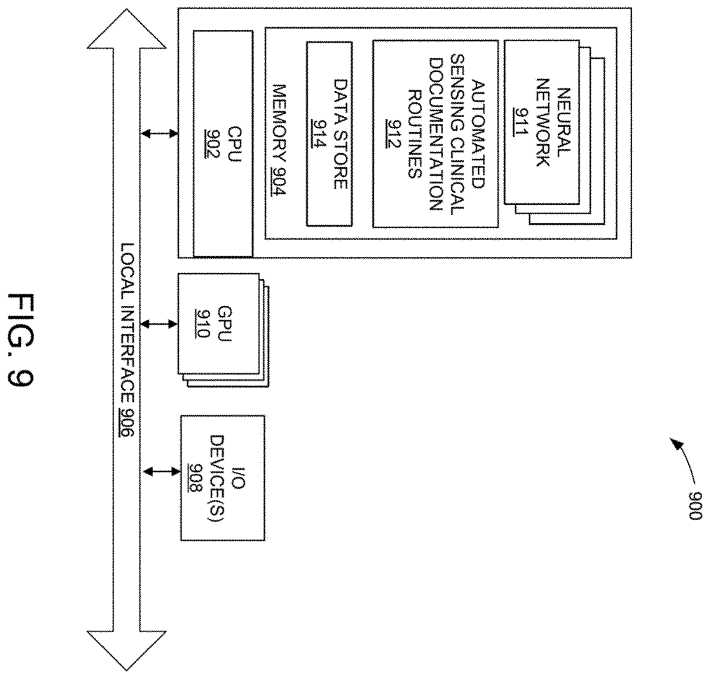

FIG. 9 provides a schematic of a computing device 900 according to one embodiment of the present disclosure. In example embodiments, the computing device 900 may be configured to train a neural network, such as a convolutional neural network, deep neural network, or the like) for automated sensing clinical documentation. An exemplary computing device 900 includes at least one processor circuit, for example, having a processor (CPU) 902 and a memory 904, both of which are coupled to a local interface 906, and one or more input and output (I/O) devices 908. The local interface 906 may comprise, for example, a data bus with an accompanying address/control bus or other bus structure as can be appreciated. The computing device 900 further includes Graphical Processing Unit(s) (GPU) 910 that are coupled to the local interface 906 and may utilize memory 904 and/or may have its own dedicated memory. The CPU and/or GPU(s) can perform various operations such as image enhancement, graphics rendering, image/video processing, recognition (e.g., object detection, feature recognition, etc.), image stabilization, machine learning, filtering, image classification, and any of the various operations described herein.

Stored in the memory 904 are both data and several components that are executable by the processor 902. In particular, stored in the memory 904 and executable by the processor 902 are code for implementing one or more neural networks 911 (e.g., DNNs) (or other machine learning models) and automated sensing clinical documentation routines 912, in accordance with embodiments of the present disclosure. Also stored in the memory 904 may be other software, such as OpenPose, etc., a data store 914, and/or other data. The data store 914 can include a database of stored task models, prepared clinical care records, network interface documents or code (e.g., web pages), and potentially other data. In addition, an operating system may be stored in the memory 904 and executable by the processor 902. The I/O devices 908 may include input devices, for example but not limited to, a keyboard, mouse, motion sensors 110, video cameras 120, etc. Furthermore, the I/O devices 908 may also include output devices, for example but not limited to, a printer, display, etc. Also, the I/O devices 908 may include a communication component, such as a network adapter or interface (e.g., WiFi network adapter, Bluetooth adapter, 4G wireless adapter, ethernet adapter, etc.), that allows for wired or wireless communications with external devices and networks.

Certain embodiments of the present disclosure can be implemented in hardware, software, firmware, or a combination thereof. If implemented in software, exemplary automated sensing clinical documentation logic or functionality are implemented in software or firmware that is stored in a memory and that is executed by a suitable instruction execution system. If implemented in hardware, the automated sensing clinical documentation logic or functionality can be implemented with any or a combination of the following technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

It should be emphasized that the above-described embodiments are merely possible examples of implementations, merely set forth for a clear understanding of the principles of the present disclosure. Many variations and modifications may be made to the above-described embodiment(s) without departing substantially from the principles of the present disclosure. All such modifications and variations are intended to be included herein within the scope of this disclosure.

Therefore, at least the following is claimed:

1. A method of sensing and documenting clinical care comprising:
recording, by at least one video camera, a video data feed of a patient being attended to by a medical personnel that is wearing at least one motion sensor;
capturing, by the at least one motion sensor, a motion data feed of the medical personnel as the patient is being attended to by the medical personnel;
collecting, by at least one computing device, the motion data feed and the video data feed;
analyzing, by the at least one computing device, the collected data feeds to prepare a clinical care record using machine learning algorithms, wherein the clinical care record indicates one or more clinical intervention procedures performed by the medical personnel as the patient is being attended to by the medical personnel and a location of a body space of the patient to which at least one of the clinical intervention procedures is performed; and
transmitting, by the computing device, the clinical care record to an upstream healthcare provider.

2. The method of claim 1, wherein the at least one video camera comprises a plurality of video cameras.

3. The method of claim 2, wherein each of the plurality of video cameras records an individual video data feed, the method further comprising:
selecting, by the at least one computing device, one of the individual video feeds that contains an unobstructed view of the patient and the medical personnel as the medical personnel is attending to the patient, wherein the selected video feed is used in preparing the clinical care record.

4. The method of claim 1, wherein the motion data feed measures hand movements of the medical personnel.

5. The method of claim 1, wherein the motion data feed further measures a muscular contraction of the medical personnel.

6. The method of claim 1, wherein the motion sensor is worn around at least one wrist of the medical personnel.

7. The method of claim 1, wherein the motion sensor is worn around at least one upper arm of the medical personnel.

8. The method of claim 1, wherein the video data feed is recorded and the motion data feed is captured during a transport of the patient to a facility of the upstream healthcare provider, wherein the prepared clinical care record is transmitted to the upstream healthcare provider prior to arrival of the patient at the facility of the upstream healthcare provider.

9. The method of claim 1, wherein the clinical care record comprises an injury heatmap diagram indicating one or more injury locations, a triage score indicating a status of the patient, and a list of clinical intervention procedures performed by the medical personnel on the patient.

10. The method of claim 9, further comprising:
constructing, by the at least one computing device, a geometric space relative to the patient's body and tracking the medical personnel's hands in the patient space by analyzing the video data feed;
determining, by the at least one computing device, a body region of the patient upon which the medical personnel is attending using the constructed geometric space;
computing, by the at least computing device, a time duration that hands of the medical personnel are recorded to be above the body region of the patient using the video data feed and the constructed geometric space; and
constructing, by the at least one computing device, the injury heatmap diagram based on the determined body region and the computed time duration.

11. The method of claim 1, wherein the analyzing of the collected data feeds comprises:
detecting an activity specific pattern from the motion data feed; and
classifying the detected activity specific pattern into a specific clinical intervention procedure that is performed by the medical personnel,
wherein the one or more clinical intervention procedures comprise the specific clinical intervention such that the clinical care record indicates the specific clinical intervention procedure was performed by the medical personnel.

12. The method of claim 11, wherein the activity specific pattern comprises a sinusoidal acceleration pattern within the collected motion data.

13. The method of claim 11, wherein the analyzing of the collected data feeds further comprises analyzing the video data feed and measuring a time duration that hands of the medical personnel are recorded to be above a specific quadrant of a body of the patient.

14. A system of sensing and documenting clinical care comprising:
at least one computing device having a processor and a memory, wherein the memory is configured to communicate with processor and stores instructions that, in response to execution by the processor, cause the processor to perform operations comprising:
obtaining a video data feed of a patient being attended to by a medical personnel and a motion data feed of the medical personnel as the patient is being attended to by the medical personnel;
analyzing the obtained data feeds to prepare a clinical care record using machine learning algorithms, wherein the clinical care record indicates one or more clinical intervention procedures performed by the medical personnel as the patient is being attended to by the medical personnel and a location of a body space of the patient to which at least one of the clinical intervention procedures is performed; and
transmitting the clinical care record to an upstream healthcare provider.

15. The system of claim 14, further comprising:
one or more wearable sensors that is configured to supply the motion data feed; and
one or more video cameras that are configured to record the video data feed.

16. The system of claim 15, wherein the one or more wearable sensors comprise an accelerometer or an electromyography sensor.

17. The system of claim 15, wherein the one or more video cameras comprise at least an RGB camera and a depth infrared camera.

18. The system of claim 15, wherein the at least one computing device is remote from the one or more wearable sensors and the one or more video cameras.

19. The system of claim 14, wherein the clinical care record comprises an injury heatmap diagram indicating one or more injury locations, a triage score indicating a status of the patient, and a list of clinical intervention procedures performed by the medical personnel on the patient, wherein the operations further comprise:
constructing a geometric space relative to the patient's body and tracking the medical personnel's hands in the patient space by analyzing the video data feed;
determining a body region of the patient upon which the medical personnel is attending using the constructed geometric space;
computing a time duration that hands of the medical personnel are recorded to be above the body region of the patient using the video data feed and the constructed geometric space; and
constructing the injury heatmap diagram based on the determined body region and the computed time duration.

20. The system of claim 14, wherein the analyzing of the collected data feeds comprises:
detecting an activity specific pattern from the motion data feed; and
classifying the detected activity specific pattern into a specific clinical intervention procedure that is performed by the medical personnel.
